(19) United States
(12) Patent Application Publication
(10) Pub. No.: US 2021/0116442 A1
(43) Pub. Date: Apr. 22, 2021

(54) METHOD AND SYSTEM FOR SENSING PLANT EXPRESSION

(71) Applicant: Ramot at Tel-Aviv University Ltd., Tel-Aviv (IL)

(72) Inventors: Yosi SHACHAM-DIAMAND, Tel-Aviv (IL); Adi AVINI, Tel-Aviv (IL); Richa PANDEY, Tel-Aviv (IL)

(73) Assignee: Ramot at Tel-Aviv University Ltd., Tel-Aviv (IL)

(21) Appl. No.: 16/767,221

(22) PCT Filed: Nov. 29, 2018
(86) PCT No.: PCT/IL2018/051313

§ 371 Date: May 27, 2020

Related U.S. Application Data
(60) Provisional application No. 62/591,869, filed on Nov. 29, 2017.

(51) Int. Cl. G01N 33/50; G01N 27/327

(52) U.S. Cl. CPC G01N 33/5097; G01N 27/327

(57) ABSTRACT

A sensing system comprises an electrochemical chip having an arrangement of electrodes configured for electrochemical sensing; a microfluidic system having fluidic channels leading to ports on a surface of the sensing system, for delivering to a plant part a substrate for a reporter enzyme expressed by the plant; and an attachment system for attaching the surface of the sensing system to a surface of the plant part in a manner that the fluidic ports contact the surface of the plant part.

### RELATED APPLICATION

This application claims the benefit of priority of U.S. Provisional Patent Application No. 62/591,869 filed on Nov. 29, 2017, the contents of which are incorporated herein by reference in their entirety.

### FIELD AND BACKGROUND OF THE INVENTION

The present invention, in some embodiments thereof, relates to sensing and, more particularly, but not exclusively, to a method and system for sensing plant expression.

With rapid development of agriculture, there has emerged a need for more abundant information to provide guidance, for example, for fertilizing and water decisions in fields. Persistent and timely monitoring of agricultural farmlands have shown to be increasingly valuable to crop health and resource management. For example, remote sensing satellites and airborne sensing with winged aircrafts have allowed scientists to map large farmlands and forests through acquisition of multi-spectral imagery and 3-D structural data. However, data from these platforms lack the spatio-temporal resolution necessary for precision agriculture.

Recently, sensors based technology have been used for measuring soil water content and nutrient analysis, weed control, pest and micro-organism control and plant physiology [Alexandratos, N. & Bruinsma, J. The 2012 Revision World agriculture towards 2030/2050: the 2012 revision, López, O. et al. Monitoring Pest Insect Traps using Low-Power Image Sensor Technologies. Sensors 12, 15801-15819 (2012), Gmur, S., Vogt, D., Zabowski, D. & Moskal, L. M. Hyperspectral Analysis of Soil Nitrogen, Carbon, Carbonate, and Organic Matter Using Regression Trees. Sensors 12, 10639-10658 (2012), Wilczek, A. et al. Determination of Soil Pore Water Salinity Using an FDR Sensor Working at Various Frequencies up to 500 MHz. Sensors 12, 10890-10905 (2012), Perez-Ruiz, M., Carballido, J., Agüera, J. & Rodríguez-Lizana, A. Development and Evaluation of a Combined Cultivator and Band Sprayer with a Row-Centering RTK-GPS Guidance System. Sensors 13, 3313-3330 (2013).

In these applications, the sensor does not interact with the plants or crops, and the analysis is based on the information perceived based on superficial observations and data collections.

### SUMMARY OF THE INVENTION

According to an aspect of some embodiments of the present invention there is provided a sensing system. The sensing system comprises: an electrochemical chip having an arrangement of electrodes configured for electrochemical sensing, a microfluidic system having fluidic channels leading to ports on a surface of the sensing system, for delivering to a plant part, optionally and preferably in vivo, a substrate for a reporter enzyme expressed by the plant, and an attachment system for attaching the surface of the sensing system to a surface of the plant part in a manner that the fluidic ports contact the surface of the plant part.

According to an aspect of some embodiments of the present invention there is provided a method of sensing plant expression. The method comprises attaching the system to a plant part using the attachment system, and receiving a signal generated by the electrochemical chip in response to exposure of the plant part to the substrate, thereby sensing the expression of the reporter enzyme by the plant.

According to some embodiments of the invention the surface of the sensing system is hydrophobic.

According to some embodiments of the invention the sensing system comprises a micro-chamber for holding the substrate, wherein the microfluidic system is constituted for delivering the substrate from the micro-chamber to the ports.

According to some embodiments of the invention the sensing system comprises an inlet port for filling the micro-chamber with the substrate.

According to some embodiments of the invention the sensing system comprises a controller for controlling dosage of the delivery of the substrate to the plant part.

According to some embodiments of the invention the sensing system comprises a communication system for transmitting signals generated by the electrochemical chip over a communication network.

According to some embodiments of the invention the sensing system comprises a controller for receiving control signals over the communication network via the communication system and controlling dosage of the delivery of the promoter substance to the plant part, based on the control signals.

According to some embodiments of the invention the electrodes are deposited on the surface of the sensing system such that the when the surface of the sensing system is attached to the surface of the plant part, the electrodes contact the surface of the plant part.

According to some embodiments of the invention the electrodes are beneath the surface of the sensing system, and wherein the microfluidic system is constituted to deliver the reporter enzyme from the ports to the electrochemical chip.

According to an aspect of some embodiments of the present invention there is provided a sensing system. The sensing system comprises: an electrochemical chip having an arrangement of electrodes configured for electrochemical sensing, a microfluidic system having fluidic channels leading to ports on the surface, for receiving from the plant part, optionally and preferably in vivo, a reporter enzyme and delivering the reporter enzyme to the electrochemical chip, and an attachment system for attaching the surface of the sensing system to a surface of the plant part in a manner that the fluidic ports contact the surface of the plant part.

According to an aspect of some embodiments of the present invention there is provided a plant or part thereof comprising a sensing system attached thereto, wherein the sensing system is as delineated above and optionally and preferably as further exemplified below.

According to an aspect of some embodiments of the present invention there is provided a method of sensing plant expression. The method comprises attaching the system as delineated above and optionally and preferably as further exemplified below to a plant part using the attachment system, and receiving signals generated by the electrochemical chip in response to exposure of the electrodes to the reporter enzyme, thereby sensing the expression of the reporter enzyme by the plant.

According to some embodiments of the invention the reporter enzyme is under the transcriptional regulation of a regulatory element. According to some embodiments of the invention the regulatory element is induced by abiotic or biotic stress. According to some embodiments of the invention the reporter enzyme is heterologously expressed in the plant or part thereof.

According to an aspect of some embodiments of the present invention there is provided a method of detecting a plant phenotype. The method comprises subjecting the plant which comprises the sensing system to a stress condition of interest that is sensed by the regulatory element, and sensing expression of the enzyme in response to the stress, the expression being indicative of the plant phenotype.

According to some embodiments of the invention the surface of the sensing system is hydrophobic.

According to some embodiments of the invention the sensing system comprises a communication system for transmitting signals generated by the electrochemical chip over a communication network.

According to some embodiments of the invention the attachment system comprises at least one mechanical assembly selected from the group consisting of a clamp, a hook and loop and a snap.

According to some embodiments of the invention the attachment system comprises an adhesive layer on the surface of the sensing system.

According to some embodiments of the invention the electrochemical chip is flexible.

According to some embodiments of the invention the electrochemical chip is attached to a surface the microfluidic system.

According to some embodiments of the invention the electrodes are deposited on a surface the microfluidic system.

According to some embodiments of the invention the electrochemical chip and the microfluidic system form a monolithic structure.

According to some embodiments of the invention the plant part is not isolated plant cells. According to some embodiments of the invention the plant part is a leaf. According to some embodiments of the invention the plant part is a stem. According to some embodiments of the invention the plant part is a bud. According to some embodiments of the invention the plant part is a root.

According to some embodiments of the invention the reporter enzyme is any unique enzyme that is not naturally occurring in the plant.

According to some embodiments of the invention the reporter enzyme is naturally occurring in the plant in a lower concentration and overtly expressed by external stimulation.

According to some embodiments of the invention the reporter enzyme is a beta glucoronidase.

According to some embodiments of the invention the substrate is selected from the group consisting of a glucuronide, Phenolphthalein-β-glucoronide and 4-aminophenyl-β-glucoronide.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration of enzyme reaction inside the cell and oxidation of the enzyme product onto electrodes surface.

FIG. 2 is a is a schematic illustration of a sensing chip, according to some embodiments of the present invention.

FIG. 3 is a graph showing measured background signal generated the sensing chip for an experimental solution containing a substrate and media in which cells were cultured, as obtained in experiments performed according to some embodiments of the present invention. Shown is cyclic voltammetry from −0.8 to 0.8V with a scan rate of 100 mV/s in the presence of 0.1M Phosphate Buffer(PB), PB+media(M), Pb+0.1M substrate(S), PB+0.1M GUS enzyme (E). Where, I/Current (Ampere) and Ewe/working electrode potential (Voltage) maintain.

FIG. 4A is an I-V graph obtained for wild type cells in experiments performed according to some embodiments of the present invention. Shown is cyclic voltammetry with wild type cells with substrate (WT+S), Transgenic cells with substrate (GUS+S).

FIG. 4B is an I-V graph obtained for commercial phenolphthalein in experiments performed according to some embodiments of the present invention. Shown is cyclic voltametry with cells in PB (PB+cells) and commercial Phenolphthalein (product of enzyme) in PB (PB+P).

FIGS. 5A-D are graph showing results of experiments performed according to some embodiments of the present invention for different substrates and stirring frequency of about 10 Hz. Shown is chronoamperometry at 700 mV (50 ul) old GUS positive cell culture with different substrate concentration from 0.5 to 10 mM. The insert shows δI Vs substrate concentration graph.

FIGS. 6A-D are images, captured during experiments performed according to some embodiments of the present invention, of leaves injected with substrate solution made in 0.1M PB pH 7.2 (phenolphthalein beta d glucuronide). Shown are images of substrate injection on the backside of the plant using a syringe (FIG. 6A), measurement sites on the GUS positive plant (FIG. 6B), spread of the substrate on the leaf (FIG. 6C), and site of measurement on the GUS negative (control) plant (FIG. 6D).

FIGS. 7A and 7B are images, captured during experiments performed according to some embodiments of the present invention, of a chip was mounted on the site of substrate injection using PDMS and a clip. The chip is connected to a portable potentiostat.

FIG. 8 is a graph showing a real time electrochemical response measured according to some embodiments of the present invention in wild type and transgenic tobacco plant. Shown is chronoamperometry at 700 mV Vs Ag/AgCl for GUS positive (GUS+) with substrate (GUS+/S) and without substrate (GUS+/PB), Gus negative (GUS−) with substrate (GUS−/S) and without substrate (GUS−/PB), Insert (Gus+/S for four different chips).

FIG. 9A is a schematic illustration showing a reduction mechanism of p-nitrophenol on electrodes surface, according to some embodiments of the present invention;

FIG. 9B is a cyclic voltammogram of 0.5 mM Enzyme GUS, 2 mM substrate p-nitrophenol beta glucoronide, substrate and enzyme together, obtained in experiments performed according to some embodiments of the present invention and demonstrating the mechanism illustrated in FIG. 9A;

FIG. 10A is a graph describing chronoamperometry of HSP+ and HSP− cells at −0.4V in the presence of different concentration of pNPG, as obtained in experiments performed according to some embodiments of the present invention;

FIG. 10B is a calibration chart ΔI vs. C concentration of pNPG, as obtained in experiments performed according to some embodiments of the present invention (ΔI calculated from the different in the current from the background current;

FIG. 11A is a graph describing chronoamperometry of HSP+ and HSP− cells at −0.4V in the presence of different concentration of PhG, as obtained in experiments performed according to some embodiments of the present invention;

FIG. 11B is a calibration chart ΔI vs. C concentration of pNPG, as obtained in experiments performed according to some embodiments of the present invention (ΔI calculated from the different in the current from the background current);

FIGS. 12A-G are a schematic illustration of an exemplary sensing system suitable for various embodiments of the present invention; and

FIGS. 13A-C are a schematic illustration of another exemplary sensing system suitable for various embodiments of the present invention.

### DESCRIPTION OF SPECIFIC EMBODIMENTS OF THE INVENTION

The present invention, in some embodiments thereof, relates to sensing and, more particularly, but not exclusively, to a method and system for sensing plant expression.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Some embodiments of the present invention comprise a sensing system that allows sensing plant functions, such as, but not limited to, stress due to an external excitation. In some embodiments of the present invention the sensing system is attached to a plant part to form a cyborg composed of the plant and sensing system. In some embodiments of the present invention the sensing system is integrated on a flexible substrate that allows in situ matching of the shape of the sensing system to the shape of the plant part to which it is attached. For example, the sensing system can be in the form of a “band aid” that can be used in the field at low cost by non-skilled personal. The sensing system can optionally and preferably communicate over a communication network, such as, but not limited to, the internet, to allow the system to transmit information to a remote location, such as a cloud computing facility and/or a cloud storage facility, for further analysis and decision making. Signals can be transmitted, e.g., over the same communication network, to a fertilization and/or irrigation system, for controlling the fertilization and/or irrigation based on the analysis of the signals from the sensing system.

In some embodiments of the present invention the communication is bilateral wherein the sensing system also receives control signals over the communication network. The control signals can include signals for delivering a promoter to the plant as further detailed hereinbelow.

The attachment of the sensing system of the present embodiments to the plant part allows electrochemical sensing of the plant biology direct from the plant. The biological information is provided by sensing electrochemically active molecules that are expressed by the reporter parts of the plant genome in response to the promoter which is the sensing part in the plant genome. The sensing system thus uses the plant itself as the sensing element wherein the electrochemical sensing is used for convert the sensing into an electrical signal. The electrical signal can then be preprocessed and optionally and preferably converted to digital signal for further digital signal processing, storage and communication.

The injection can be regulated using a microfluidic system organized in a ring shape around the flexible polymer based sensing electrode. The electrodes can be made on a specially manufactured substrate, using any technique such as, but not limited to, lithography or 3D printing, matching the pore patterns on the leaves or inserted into the plant. The sensing system can be grafted onto the plants, this way the growth and maintenance of the genetically modified sensor will be separated from the actual plant which is not to be considered as a GMO.

In some embodiments, the plant expresses the substrate material and there is no need for an external injection of the substrate. The reporting gene can be integrated with a promoter gene detecting many desired phenomena that affect the plant. Promoter genes can be integrate on the plants. Alternatively, existing plant promoter genes can be used.

The sensing system of the present embodiments can be used for sensing external conditions such as, but not limited to, temperature, water content, nutrients, pesticides, and/or internal parameters such as, but not limited to, plant hormones serving as signal transmitters, e.g., Jasmonic acid or Methyl Jasmonate. The sensing system of the present embodiments can detect physical parameters as well as monitor the information generated inside the plants and communicate between plants.

The sensing system of the present embodiments can be part of an internet of things (IoT) network, and thus be used for collecting data over a large number of plants, over a large area, for a long time, and at low cost. This allows precise agriculture regarding all stages of planing, growth, harvesting, storage, and distribution directly or indirectly, for example, using further processing to the point of sale. The sensing system of the present embodiments can be used as a point of care application of water, nutrients and pesticides, storage monitoring and food quality forecasting.

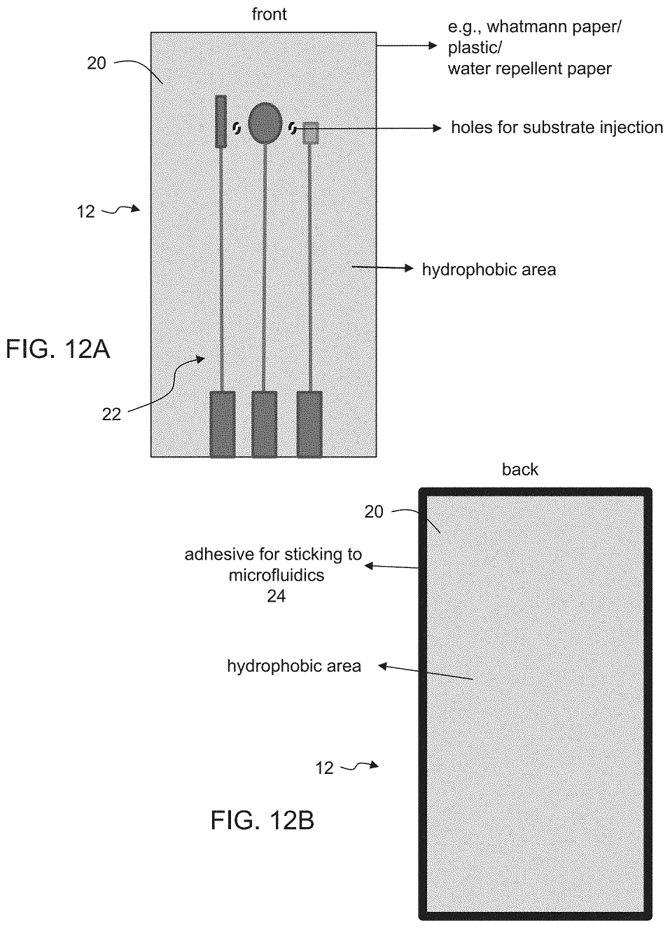

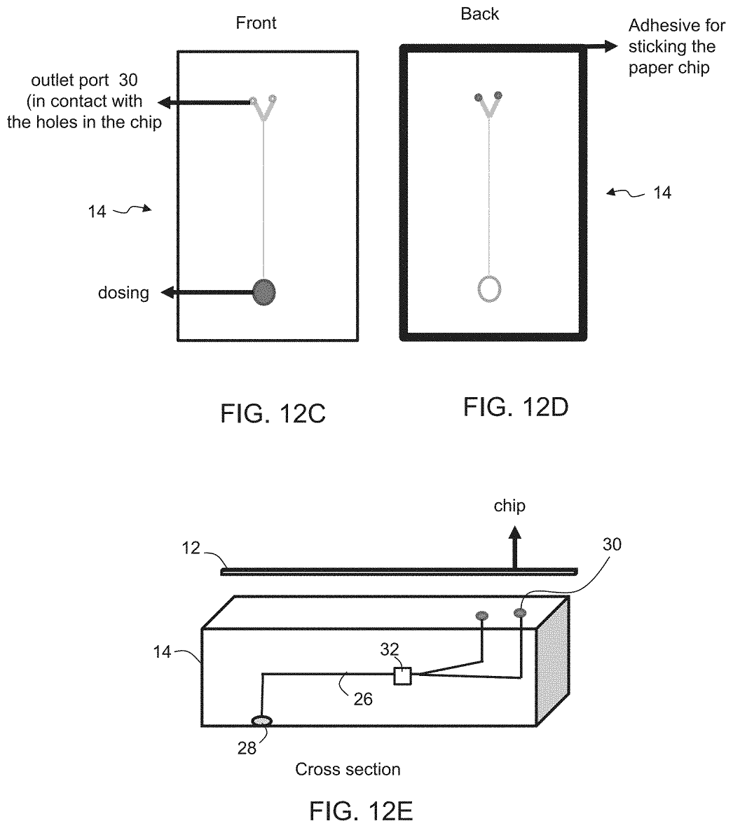

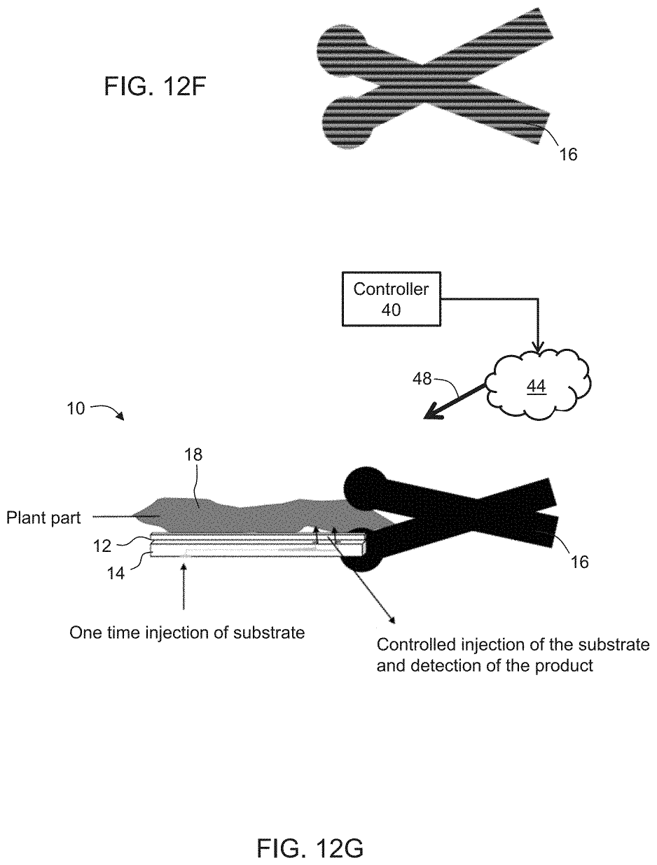

A representative illustration of a sensing system 10 suitable for various exemplary embodiments of the present invention is provided in FIGS. 12A-G. The sensing system 10 of these embodiments comprises an electrochemical chip 12 for detecting an electrochemical signal, a microfluidics system 14 for introducing the substrate into a plant part 18 (e.g., a leave, a stem, a bud, a root), and an attaching system 16 for applying pressure on the plant part 18 (e.g., the top part of the plant part) and for holding the system.

The term “microfluidic system” as used herein refers to a system having one or more fluid microchannels.

The term “microchannel” as used herein refers to a fluid channel having cross-sectional dimensions the smallest of which being less than 1 mm, more preferably less than 500 μm, more preferably less than 400 μm, more preferably less than 300 μm, more preferably less than 200 μm, e.g., about 100 μm or about 10 μm.

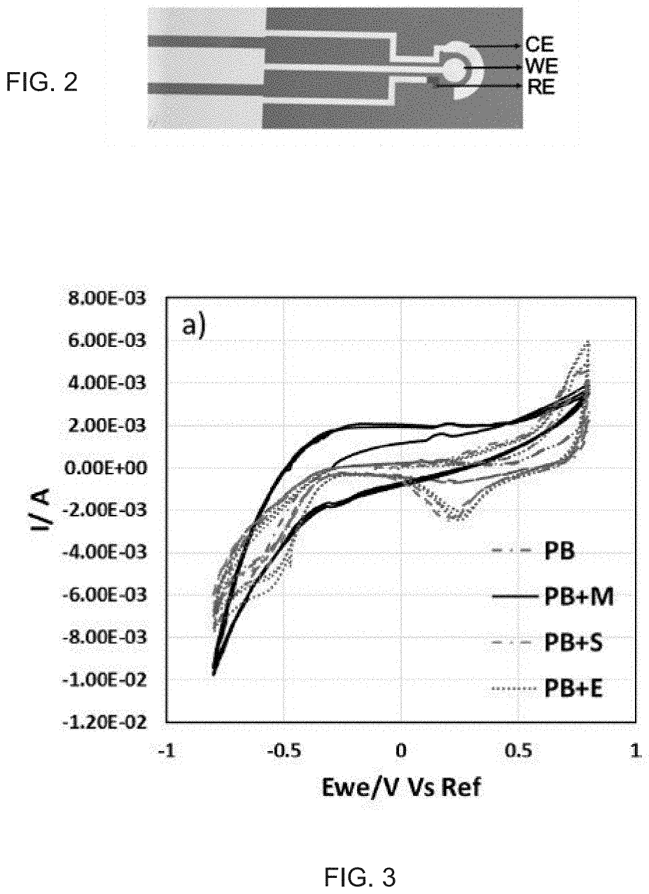

FIGS. 12A and 12B are schematic illustrations of a front view (FIG. 12A) and a back view (FIG. 12B) of the electrochemical chip 12. Chip 12 comprises a solid structure 20 formed with a plurality of electrodes 22 arranged for electrochemical sensing. For example, electrodes 22 can include a reference electrode (RE), a working electrode (WE), and a counter electrode (CE). The working electrode is the electrode at which the electrochemical reaction occurs. Depending on the type of reaction, the working electrode can serve as a cathode or as an anode. Suitable materials for the working electrode including, without limitation, carbon (e.g., glassy carbon, activated carbon cloth, carbon felt, platinized carbon cloth, plain carbon cloth), gold, platinum, silver and the like. The counter electrode is optionally and preferably, but not necessarily, made of the same material as the working electrode. The reference electrode can be a Silver/Silver Chloride electrode, a calomel (e.g., saturated calomel) electrode, or the like. A representative arrangement of RE, WE and CE is illustrated in FIG. 2.

Solid structure 20 is optionally and preferably flexible. In various exemplary embodiments of the invention structure 20 has a hydrophobic surface that may encompass the entire or part of the area of structure 20. The hydrophobic surface is optionally and preferably at both the front and the back sides of structure 20.

The term “hydrophobic”, as used herein, refers to a trait of a molecule or part of a molecule which is non-polar and is therefore immiscible with charged and polar molecules, and has a substantially higher dissolvability in non-polar solvents as compared with their dissolvability in water and other polar solvents. The phrase “dissolvability” refers to either complete dissolution of the substance in these solvents or to cases where the substance reaches its maximal saturation concentration in non-polar solvents, and the remainder of the substance is in the form of a suspension of small solid particles in the solvent. When in water, hydrophobic molecules often cluster together to form lumps, agglomerates, aggregates or layers on one of the water surfaces (such as bottom or top). Exemplary hydrophobic substances suitable for the present embodiments include, without limitation, substances comprising one or more alkyl groups, such as oils and fats, or one or more aromatic groups, such as polyaromatic compounds. For example, the structure 20 can be made of, or comprise, a substrate selected from the group consisting of a paper, e.g., a Whatman® Cellulose Filter Paper.

The back side of structure 20 optionally and preferably comprises an adhesive 24 for facilitating attachment of the back side of structure 20 to microfluidics system 14.

FIGS. 12C-E are schematic illustrations showing the microfluidic system 14 according to some embodiments of the present invention. System 14 comprises one or more fluidic microchannels 26. Microchannels 26 can include a linear microchannel extending along a generally (e.g., within deviation of 10% or less) straight line, or a nonlinear microchannel, in which case at least part of microchannels 12 extends along a curved line. Microchannels 26 can alternatively or additionally include a nonlinear microchannel, in which case at least part of the microchannel extends along a curved line a plurality of interconnected segments. Microchannels 26 can alternatively or additionally include a plurality of interconnected segments. These embodiments include a configuration in which all the segments are linear, or configuration in which all the segments are nonlinear, or a configuration in at least one of the segments is linear and at least one of the segments is nonlinear.

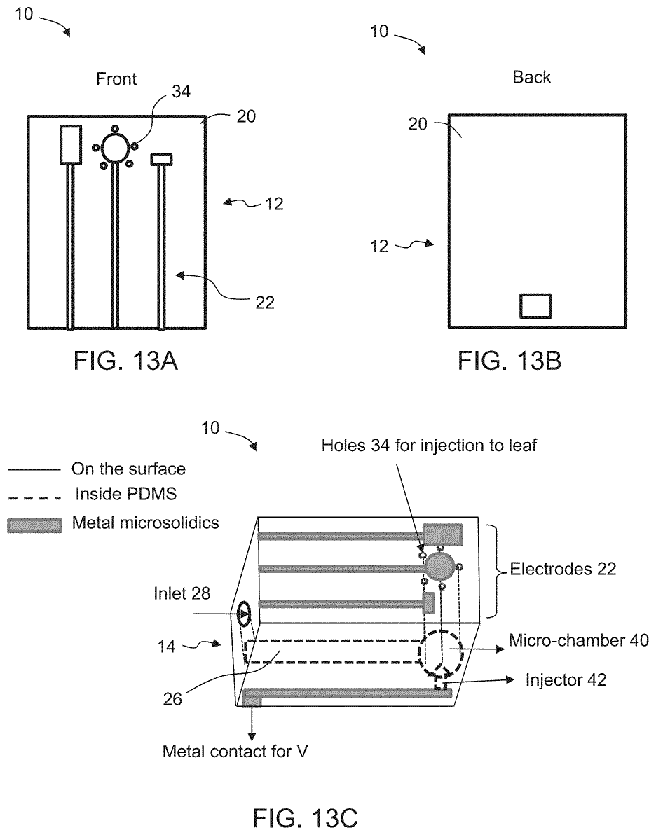

Microfluidic system 14 typically comprises one or more inlet ports 28 and one or more outlet ports 30. Inlet port 28 serves for receiving liquid, such as, but not limited to, a substrate for a reporter enzyme. The liquid can be introduced to port 28, for example, by means of a syringe or the like. Preferably, but not necessarily, the liquid is introduced to port 28 as a one-time (not repeated) operation. In some embodiments of the present invention system 14 comprises a micro-chamber (not shown, see FIG. 13C) for holding the received liquid.

Outlet ports 30 serve for delivering the liquid (for example, from the micro-chamber, but may also be from the microchannel 26) out of system 14, preferably through chip 12 into the plant part 18. The ejection of the liquid (e.g., substrate for a reporter enzyme) can be effected by any technique known in the art, including, without limitation, concentration driven ejection, pressure driven ejection, electrothermal actuation, etc. In some embodiments of the present invention system 14 comprises a controllable valve or actuator 32 that ejects the liquid out of port(s) 30 responsively to a control signal 48 transmitted over a communication system 44 such as, but not limited to, a local area network (LAN), a wide area network (WAN) or the Internet, from a controller 46. It is to be understood, however, that ejection of liquid out of port(s) 30 can also be effected without an external signal (e.g., by means of concentration differences, or the like).

The microfluidic system 14 can be made of any material known in the art such as, but not limited to, an elastomeric polymer such as polydimethylsiloxane (PDMS), polytetrafluoroethylene (PTFE), polyisoprene, polybutadiene, polychloroprene, polyisobutylene, poly(styrene-butadiene-styrene), polyurethanes, silicones, PMMA, and polycarbonate.

Returning to FIG. 12A, chip 12 preferably comprises one or more through holes 34 that are aligned with outlet ports 30 of microfluidic system 14. Liquid contained in the micro-chamber or microchannel 26 is ejected out of port 30, passes through chip 12 via the through holes 34, and contacts the plant part 18. Optionally, but not necessarily, through holes 34 are arranged in the vicinity of one of the electrodes 22. For example, several through holes can be arranged along the periphery of the electrode (e.g., along the periphery of the working electrode).

FIG. 12F is a schematic illustration of attachment system 16 according to some embodiments of the present invention. The attachment system 16 holds the system 10 attached to the plant part 18, and optionally and preferably also applies pressure on the front side of the plant part 18 for easy diffusion for the substrate into the plant part. The attachment system in FIG. 12F is illustrated as a clamp, but other mechanisms are also contemplated. The contact area of the clamp is optionally and preferably slightly larger than the circular area around the electrode. The sensing system, once attached to a plant part (a leaf, in the present Example) is illustrated in FIG. 12G.

The electrodes 22 of chip 12 can be formed on structure 20, by printing, deposition, sputtering, evaporation or the like. Holes 34 can be drilled through the structure 20 at the vicinity of one or more of the electrodes (e.g., the working electrode) for allowing the substrate to pass through the chip 12 into the plant part. This can be done, for example, using a cutter or a puncture device.

The hydrophobic area can be applied to structure 20 in more than one way. One way is by masking and dipping. Specifically, a mask is used to cover the electrode area, the structure 20 is then dipped into a bath, which can be filled with a hydrophobic liquid (e.g., a commercially available wax material), and is then heated in oven. The mask is removed, and a different mask, preferably complementary to the first mask, is used to cover the hydrophobic area. The electrodes are then formed on the unmasked and non-hydrophobic surface.

Another way is by wax printing. In this technique a wax ink is selectively applied to the substrate using a printer.

The hydrophobic area can alternatively include a water repellent paper. In these embodiments, the electrodes can be directly deposited onto the paper, for example, using a shadow mask.

The hydrophobic area can alternatively be made of a polymeric material plastic, such as, but not limited to, polyimide, polystyrene or any other thin polymeric film. The electrodes can be directly deposited onto the polymeric material using a mask.

Another representative illustration of sensing system 10 suitable for various exemplary embodiments of the present invention is provided in FIGS. 13A-C. In this exemplified configuration, which is not to be considered as limiting, the electrochemical chip 12 and microfluidic system 14 optionally and preferably form a monolithic structure having also a micro-chamber 40 for holding the liquid and an injector 42 for injecting the liquid into through holes 34. The monolithic structure can be made of any of the materials specified above with respect to the microfluidic system 14 illustrated in FIGS. 12A-G. The monolithic structure can be fabricated using any technique such as, but not limited to, molding and microsolidics technique. A master including the features of electrodes 22, the channels 26, and the injector 42 can be fabricated, for example, by rapid prototyping. Thereafter, molding and curing can be applied using the selected elastomeric polymer. A silinisation can be applied on the electrode area for next step of microsolidics. A molten solder can be introduced into the channels by applying a vacuum to draw metal into the channels. The walls of the silanized channels can be rapidly wet by liquid solder. The channels can then be cooled to form solid metal microstructures.

In some embodiments of the present invention electrodes 22 are beneath the surface of the sensing system 10, so that there is no contact between electrodes 22 and plant part 18. In these embodiments, microfluidic system 14 has ports on the surface of system 10 (such as ports 30 except that the electrodes are beneath the surface. In these embodiments, microfluidic system 14 has delivers the reporter enzyme via the surface ports 30 to electrochemical chip 12.

The term “plant” as used herein encompasses whole plants, a grafted plant, ancestors and progeny of the plants and plant parts, including seeds, shoots, stems, roots (including tubers), rootstock, scion, and plant cells, tissues and organs. The plant may be in any form including suspension cultures, embryos, meristematic regions, callus tissue, leaves, gametophytes, sporophytes, pollen, and microspores. Plants that are particularly useful in the methods of the invention include all plants which belong to the superfamily Viridiplantae, in particular monocotyledonous and dicotyledonous plants including a fodder or forage legume, ornamental plant, food crop, tree, or shrub selected from the list comprising Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Cannabis indica, Hemp, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalypfus spp., Euclea schimperi, Eulalia vi/losa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lotonus bainesli, Lotus spp., Macrotyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canariensis, Phormium cookianum, Photinia spp., Picea glauca, Pinus spp., Pisum sativam, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, trees. Alternatively algae and other non-Viridiplantae can be used for the methods of some embodiments of the invention.

Constructs useful in the methods according to some embodiments of the invention may be constructed using recombinant DNA technology well known to persons skilled in the art. The gene constructs may be inserted into vectors, which may be commercially available, suitable for transforming into plants and suitable for expression of the gene of interest in the transformed cells. The genetic construct can be an expression vector wherein said nucleic acid sequence is operably linked to one or more regulatory sequences allowing expression in the plant cells.

In a particular embodiment of some embodiments of the invention the regulatory sequence is a plant-expressible promoter.

As used herein the phrase “plant-expressible” refers to a promoter sequence, including any additional regulatory elements added thereto or contained therein, is at least capable of inducing, conferring, activating or enhancing expression in a plant cell, tissue or organ, preferably a monocotyledonous or dicotyledonous plant cell, tissue, or organ. Examples of preferred promoters useful for the methods and systems of some embodiments of the invention are provided here: heat shock promoter 18.2 and RD29 for detection drought. Others include: PR00151 (WSI18 which is active in embryo and stress), PR00175 (RAB21, which is active in embryo and stress).

Also contemplated are promoters selected from the group consisting of salicylic acid inducible promoter, tetracycline inducible promoter, and ethanol inducible promoter. Further contemplated are promoters selected from the group consisting of Cor78, Corl5a, Rci2A, Rd22, cDet6, ADH1, KAT1, KST1, Rhal, ARSK1, PtxA, SbHRGP3, GH3, the pathogen inducible PRPl-gene promoter, the heat inducible hsp80-promoter from tomato, cold inducible alpha-amylase promoter from potato, the wound-inducible pinU-promoter.

According to a specific embodiment the reporter enzyme is a β-glucuronidase (EC 3.2.1.31) an acid hydrolase enzyme that cleaves a wide variety of β-glucuronic acids.

Plant cells may be transformed stably or transiently with the nucleic acid constructs of some embodiments of the invention. In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225; Shimamoto et al., Nature (1989) 338:274-276).

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 679:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledenous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by some embodiments of the invention.

Transient transformation can be effected by any of the direct DNA transfer methods described above or by viral infection using modified plant viruses.

Viruses that have been shown to be useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself.

Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of some embodiments of the invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

The systems described herein for determining expression in plants, can be used to identify the condition of the plant, for instance, whether the plant is grown under stress or non-stress conditions.

As used herein the term “about” refers to ±10%.

The word “exemplary” is used herein to mean “serving as an example, instance or illustration.” Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments.” Any particular embodiment of the invention may include a plurality of “optional” features unless such features conflict.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### In Vivo Electrochemical Sensing

This Example demonstrates the continuous and real time monitoring of the expression of the β-glucuronidase (GUS) enzyme in transgenic tobacco plant, by sensing of GUS enzyme in Msk8 tomato cells.

The prime applications of sensors in the agriculture today is in soil water content and nutrient analysis, weed control, pest and micro-organism control and plant physiology. However, current approaches are indirect, where the sensor doesn't interact with the plants or crops and rely on the information perceived based on the superficial observations and data collections.

In this Example, the expression of the enzyme β-glucuronidase (GUS) was studied first in the cells of the transgenic tomato plant and then in a transgenic tobacco plant by integrating them with an electrode chip. β-glucuronidase (EC 3.2.1.31) is an acid hydrolase enzyme that cleaves a wide variety of β-glucuronic acids. The GUS was selected for this Example since this enzyme does not result in a background signal in the plant, and since its substrates are commercially available.

The following shows that electrochemical approach can be used to assay the activity of GUS in Msk8 tomato cells using Phenolphthalein-β-glucuronic acid as the substrate. This example also demonstrate a real time in-vivo detection of GUS enzyme expressed by transgenic tobacco plants by integrating a three electrode chip with the plant using a portable potentiostat.

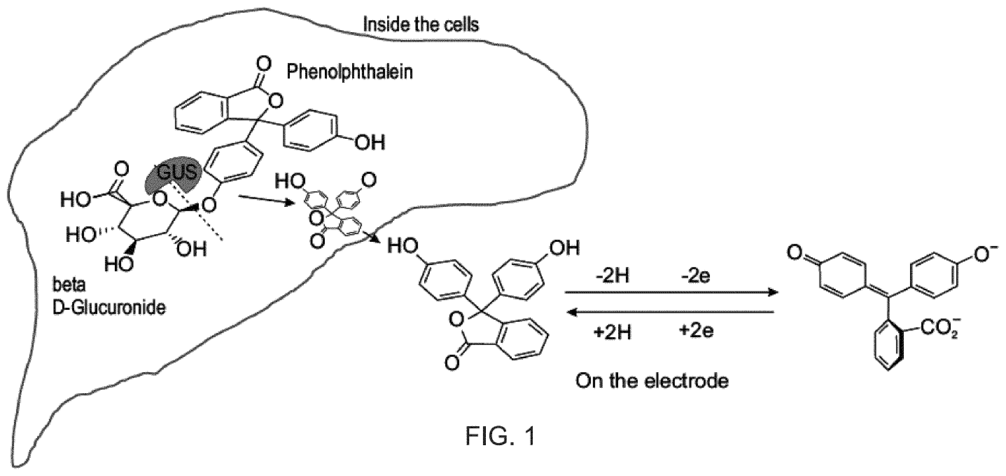

FIG. 1 demonstrates the mechanism of enzyme and substrate reaction inside the cells. Without wishing to be bound to any particular theory, it is assumed that the phenolphthalein is taken up by the cells where the GUS catalyzes its cleavage into phenolphthalein and glucuronic acid. The phenolphthalein traverses out and gets oxidized by removal of an electron on the electrode to at 0.7V. To test the sensing platform (FIG. 2), the background signal generated by an experimental solution containing the substrate and the media in which the cells were cultured was measured (FIG. 3).

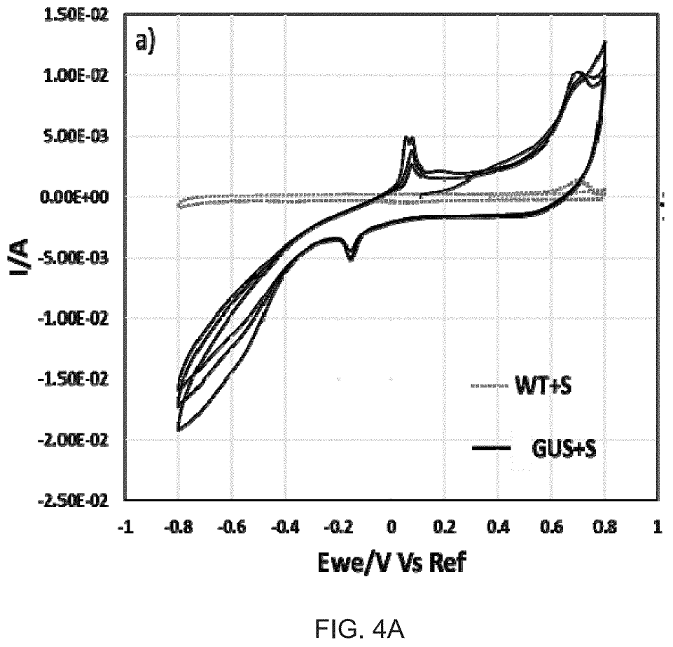

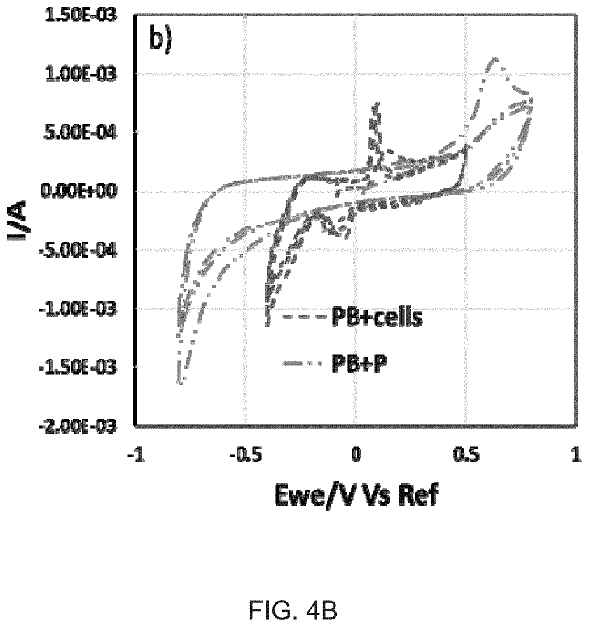

None of these components were electroactive and did not interfere with the plants signal. The cyclic voltammogram showed no significant peaks for the enzyme, substrate, media and the background solution which was phosphate buffer in this Example. The control experiment was with wild type (WT) cells which did not express the enzyme. In the CV (FIG. 4A) the WT cells with substrate gave very little or negligible oxidation at around 0.7V. However, the GUS positive cells gave a higher current response of about 0.1 mA than WT. To confirm that this oxidation peak was due to oxidation of the phenolphthalein, a cyclic voltammetry of commercial phenolphthalein was performed on the same chip (FIG. 4B). This figure demonstrates that the oxidation potential of the phenolphthalein is 0.7 and is coherent with the experiment that performed with cells. A reversible redox peaks at 0.15 and −0.15V (FIG. 4A) in the CV of GUS cells with the substrate was also observed. No presence of these peaks was observed in any other CVs. Another CV of GUS cells with PB was performed to investigate the presence of the redox peaks. Similar peaks were observed so that the cells are probably producing or have some electroactive component that is likely to undergo a redox reaction at these near zero potentials (FIG. 4B).

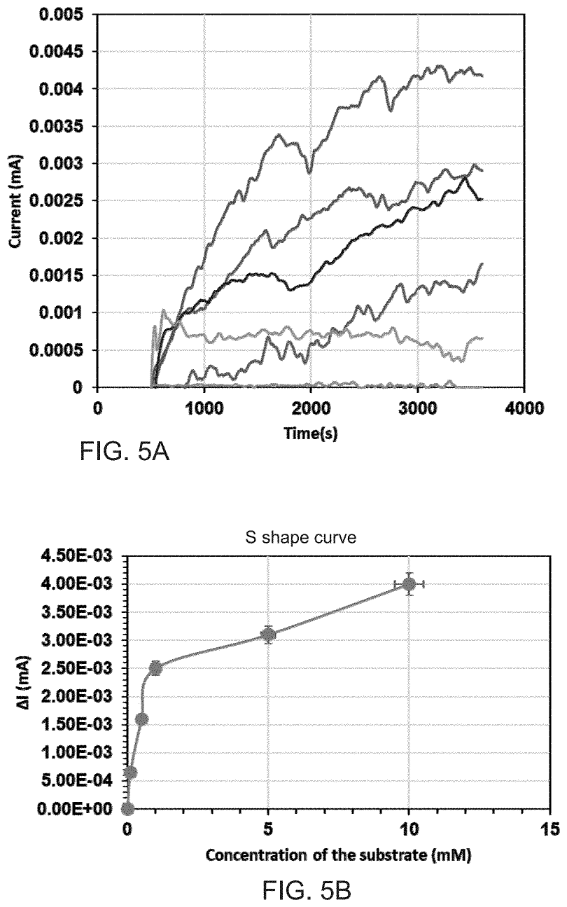

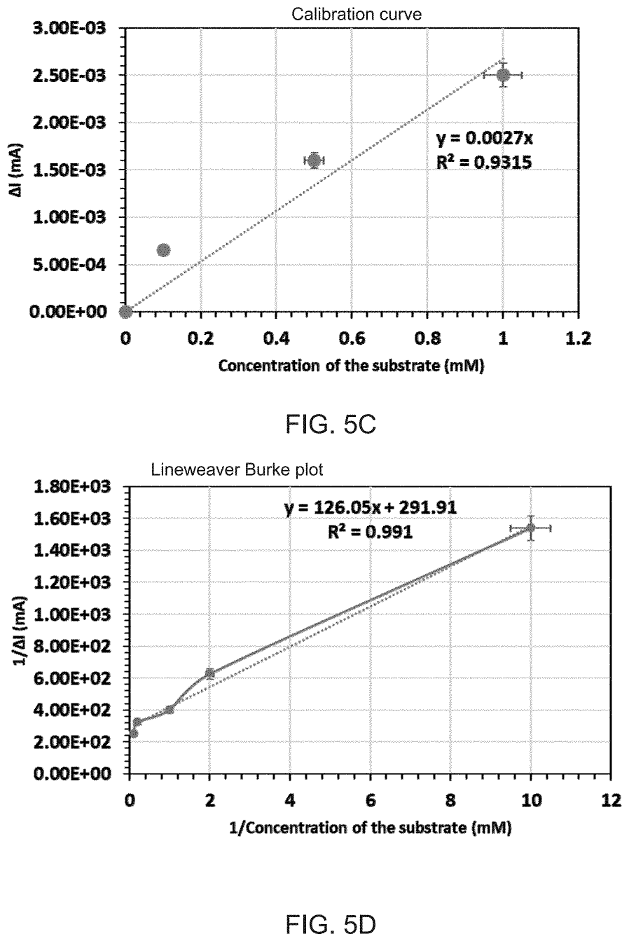

The current response of the chip containing GUS positive cells with different substrate concentration for a duration of the 2500 s was studied (FIG. 5A). There was a steady increase in the current signal after the substrate was added at 500 s. However, the response was not spontaneous and took about 100 s after to observe an increase in the current. In control experiment, where the substrate was not added, the chronoamperogram showed a negligible increase in the current. A calibration graph was plotted by calculating the ΔI (current at 400 s subtracted from the current at the 2500 s) with different substrate concentration (FIGS. 5B and 5C). The typical S shape curve was observed dictating to Michaelis Menten equation. The straight region of the calibration curve was used to calculate the sensitivity of 2.7 μA/mM and the limit of detection (LOD) of 10 μM. The data of calibration graph was also plotted as the Lineweaver-Burke plot (FIG. 5D), and the Vmax and Km were calculated to be 0.34 μA and 0.43 mM respectively. The Km for Gus obtained from calf liver is known to be 0.148 mM. For the GUS enzyme of E. coli origin, the Km is known to be in the range of 0.018 to 3.05 mM for Phenolphthalein glucoronide.

Additional experiments were conducted to demonstrate the ability of the present embodiments to sense the expression of the same enzyme in whole plants. Unlike conventional techniques, the the present Inventors successfully measured a realtime electrochemical response from a plant by integrating a sensing chip to the leaves.

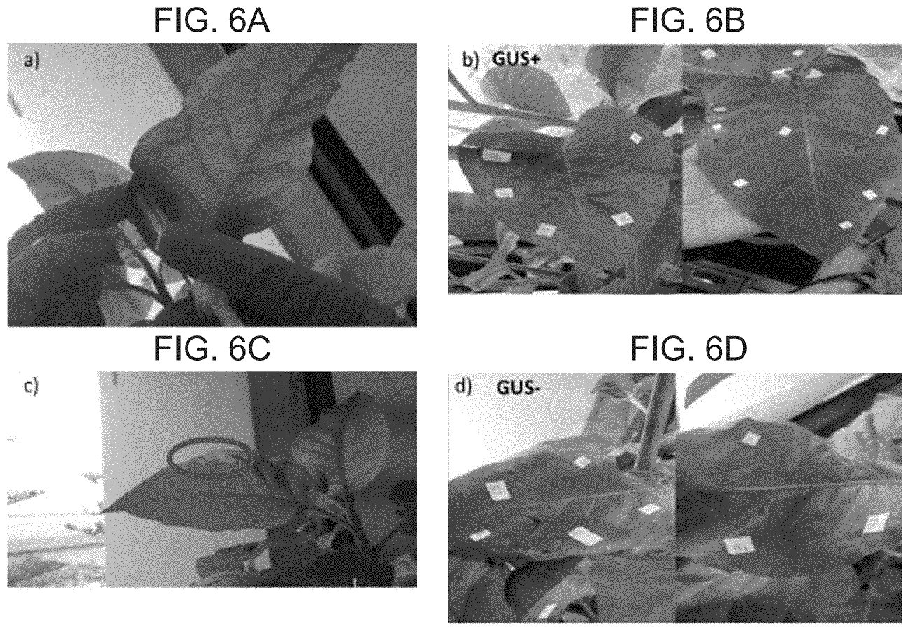

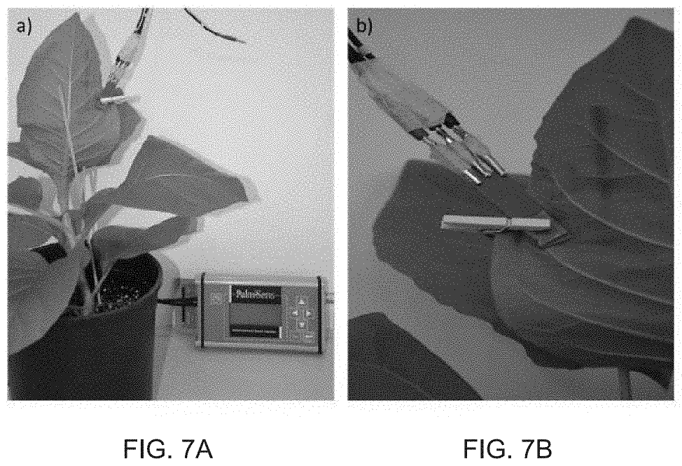

0.1 ml of 1.2 mg/ml of substrate solution made in 0.1M PB pH 7.2 (phenolphthalein beta d glucuronide) was injected at the back side of the leaf using a syringe (FIGS. 6A-D). The solution entered through stomata and diffused in the intracellular matrix of the of the leaf. Blank measurements were performed by injecting 0.1 ml PB. The chip was then mounted on the site of substrate injection using PDMS and a clip (FIGS. 7A and 7B).

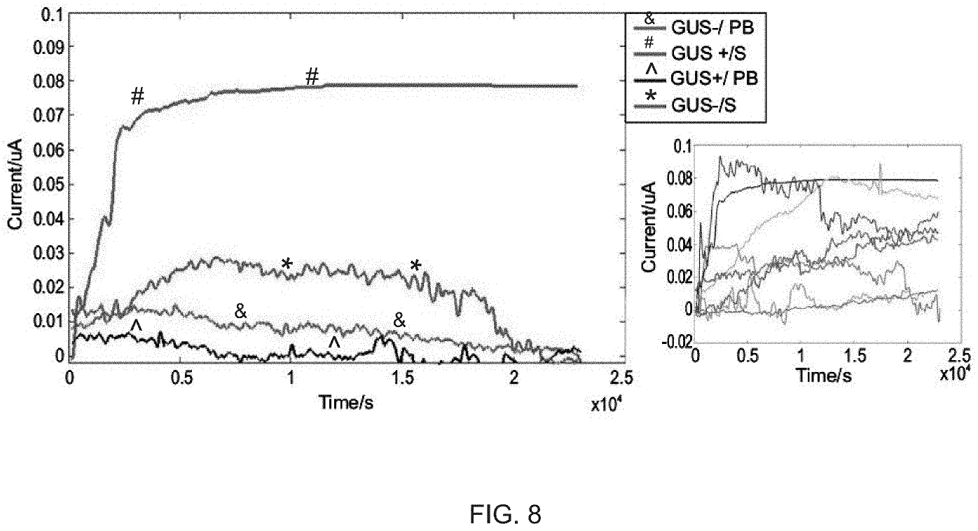

Three control experiments were performed to test if there is another chemical in the plants that gives a response in the presence of the substrate. A real time electrochemical response was measured in the wild type (control) and transgenic tobacco plant (over expressing the GUS gene) and is shown in FIG. 8. The control experiment on the transgenic and wildtype plants was performed without the substrate injection and showed a negligible current response. This assured that there are no electroactive species in the plant that can generate a strong background current that interferes with the actual current signal. Then, the realtime electrochemical response was measured in wildtype and transgenic plants after injecting the substrate (FIG. 8).

An initial increase in current with the control plant injected with the substrate was observed. The signal fastly reached steady state and then dropped. It is assumed that this behavior is a result of impurities in the substrate as explained earlier. The increase of the faradaic current due to the oxidation of the product produced by the enzyme catalyzed the reaction of the transgenic plant was found to be significant (four times) when compared to all the three control experiments. The same experiments were performed in 5 different chips and gave the similar trend (FIG. 8, inset). However, since the level of GUS expression and the extent of diffusion of the substrate adjacent to the sensor, was not under control, different levels of increment in the current signal were observed.

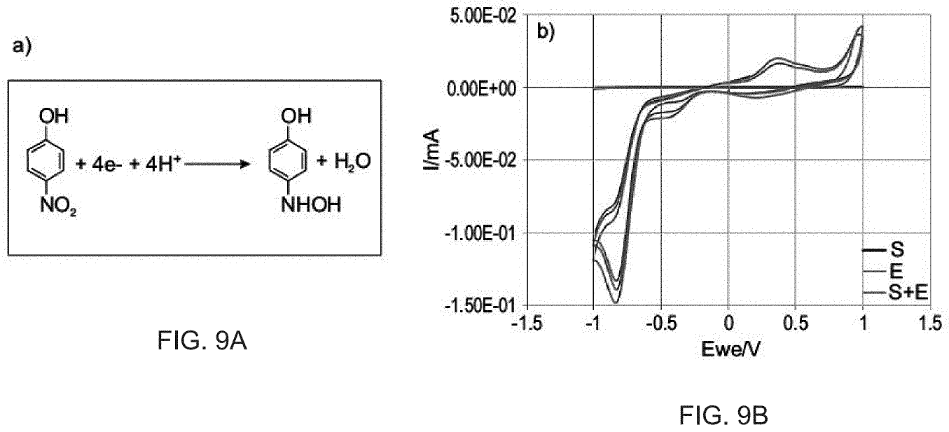

FIG. 9A demonstrates the reduction mechanism of the pNP on the electrodes surface. This can also be studied by CV as delineated in FIG. 9B. The CV (blue) of pNPG and GUS together shows no oxidation peak in the first cycle, however there is a reduction peak at −0.4V due to reduction of pNP produced by the reaction of GUS and pNPG. Just enzyme and the substrates didn't show any redox peak due to them being not electroactive.

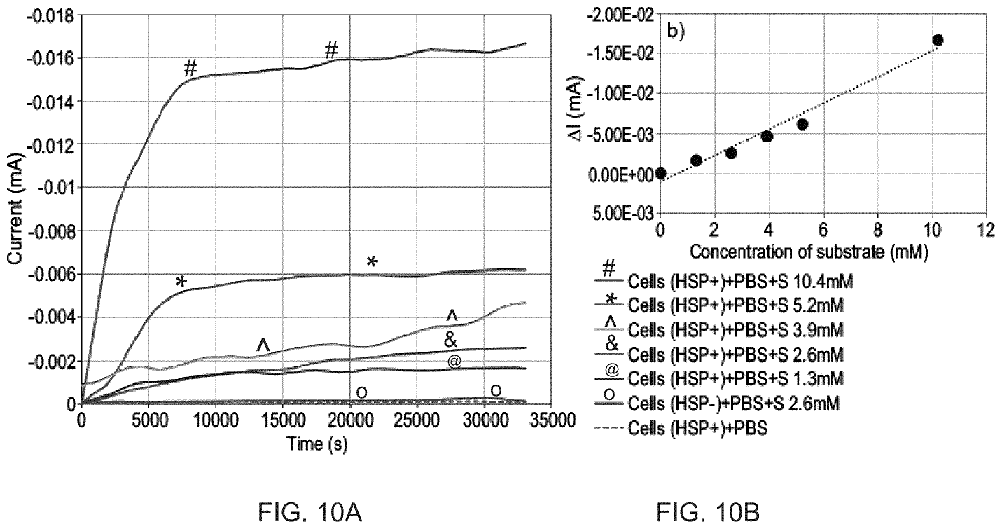

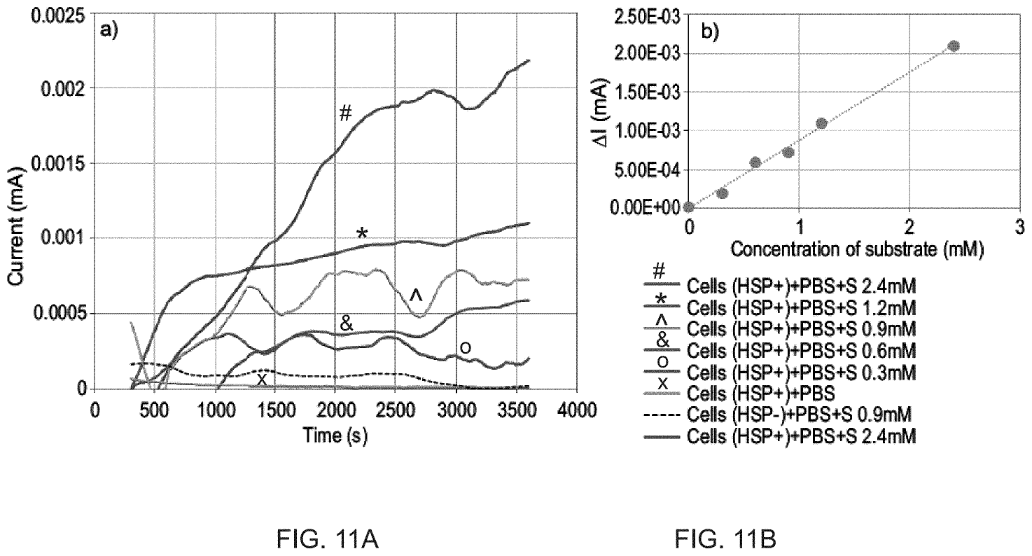

So, we performed the chronoamperometry (FIG. 10a) using the HSP+ and HSP− cells and pNPG at −0.4V vs Ag/AgCl. The HSP− cells in the presence of pNPG was our first control that didn't give any increase in the current. Our second control was HSP+ cells without pNPG, this also didn't give any increase in the current response. An increase in the current is observed with subsequent increase in the substrate concentration. This difference in the current when compared to the background current was plotted against the pNPG concentration as calibration in FIG. 10.b. We also confirmed this by using the previously used substrate PhG. The chronoamperometry (FIG. 11.a) was performed for the two control experiments as mentioned in the previous experiment and also with increasing concentration of the PhG. A linear increase in the current response was observed which was then plotted in the calibration chart (FIG. 11.b).

After the cells were treated with heat shock there was an induction based expression of GUS enzyme by the heat shock proteins. This expressed GUS is then detected by another substrate pNPG. This substrate was chosen because it reacts with enzyme and produces a product that is electroactive at −0.4V. This potential is low enough to eliminate the interference due to the hydrolysis of water when PhG was used as the substrate. The calibration chart obtained from the chronoamperogram was then used to calculate the sensitivity (2.25 A/mM-cm2) and LOD (0.6 mM). An increased sensitivity is observed here due to increased electron transfer rate between the product and the electrode. Apart from pNPG the cells were also studied with PhG same as mentioned in the constitutive GUS expression detection. The calibration obtained for different concentration of PhG demonstrated a lower sensitivity (1.23 mA/mM-cm2) than the pNPG studies.

In this Example, the ability of the sensing system optionally and preferably to sense expressed genes in plants was demonstrated. The reporting gene generated the enzyme β-glucuronidase that were detected using its interaction with a specific substrate and the oxidation of the product generated by the reaction of this enzyme and substrate. Various control experiments were performed to demonstrate that the signal obtained is from the desired enzyme-substrate reaction. A calibration chart was plotted for different concentration of the substrate to calculate the sensitivity and limit of detection of 0.27 mA/mM and 0.1 mM respectively. The Km calculated (0.6 mM) was found to be in the theoretical range as reported in the literature. The performance of the sensing system integrated with plant leaf showed a higher current response when compared to the control experiment of without the substrate and the enzyme.

### Methods

Cells and Plant Culture

Suspension-cultured cells (S. lycopersicum cv Mill.; line Msk8) were grown as described by Felix et al²¹ (Felix et al., 1991) and used 4 to 6 days after weekly sub culturing. Msk8 cells were transformed by Agrobacterium strain EHA105 harboring pBIS-N1 plasmid containing the GUS gene.

Nicotiana tabacum were transformed by Agrobacterium strain GV3101 harboring the GUS gene in the vector pPCV702 as previously described e.g., Klee et al. Ann. Rev. Plant Physiol. 1987 38:467-86; De Block et al. EMBO J. 1984 3(8):1681-1689; Timko et al. 1984 310(12):115-120.

Electrochemical Chip Fabrication

Three electrode electrochemical chips (FIG. 2) were fabricated on 4″ silicon wafers (p-Si [symbol] 1 0 0 [symbol] , with 500 nm thick thermal oxide layer, University Wafer Inc.) and in the cleanroom using a combination of photolithography and sputtering techniques. Briefly, the patterned wafer using photolithography were sputtered with a 15 nm Ti and 150 nm Au thin films without breaking the vacuum. A lift off was performed to obtain the final pattern of seven chip a wafer. Every single chip consisted of a planar gold working electrode (3.14 mm²), the gold counter electrode (6.28 mm²) and Ag/AgCl open reference electrode (1 mm²). The reference electrode was made by electroplating of Ag and later forming AgCl.

Characterization

All the electrochemical measurements were performed in 0.1M Phosphate buffer (PB) of pH 5.8 for the MSK8 cells and pH 7.1 for the Tobacco plants. All the electrochemical studies using the cells were performed using VSP BioLogic potentiostat. The cyclic voltammetry was performed on the three electrode chips for the PB (control), substrate Phenolphthalein beta-D glucuronide (PhG, 0.1M, Sigma Aldrich) and p-nitrophenyl beta-D glucuronide (pNPG, 2 mM, Sigma Aldrich), commercial enzyme Beta D-Glucuronidase (GUS 0.1M, Sigma Aldrich), product Phenolphthalein (Phe 0.1M, Sigma Aldrich), media in which cells were grown and cells with a voltage sweep from 1V to −1V with a scan rate of 100 mV/s. Next, the cyclic voltammetry was performed using the 20 μl of young/old Msk8 and wild type tomato cells in the presence of the substrate. Initial chronoamperometry study for different PhG concentration and pNPG concentration was performed at 0.7V and −0.4V vs. Ag/AgCl respectively. These potentials were selected after conducting cyclic voltammetry at the same system configuration in the presence of respective substrates. For the experiments in tobacco plants, chronoamperometry was performed by a portable Palmsense® potentiostat (Palm Instruments BV, the Netherlands) at 700 mV vs. Ag/AgCl by injecting 0.1 ml PhG from the back of the leaf.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

1. Alexandratos, N. & Bruinsma, J. The 2012 Revision World agriculture towards 2030/2050: the 2012 revision. (2030).
2. López, O. et al. Monitoring Pest Insect Traps using Low-Power Image Sensor Technologies. Sensors 12, 15801-15819 (2012).
3. Gmur, S., Vogt, D., Zabowski, D. & Moskal, L. M. Hyperspectral Analysis of Soil Nitrogen, Carbon, Carbonate, and Organic Matter Using Regression Trees. Sensors 12, 10639-10658 (2012).
4. Wilczek, A. et al. Determination of Soil Pore Water Salinity Using an FDR Sensor Working at Various Frequencies up to 500 MHz. Sensors 12, 10890-10905 (2012).
5. Perez-Ruiz, M., Carballido, J., Agüera, J. & Rodríguez-Lizana, A. Development and Evaluation of a Combined Cultivator and Band Sprayer with a Row-Centering RTK-GPS Guidance System. Sensors 13, 3313-3330 (2013).
6. Kim, G., Kim, G., Ahn, C.-K., Yoo, Y. & Cho, B.-K. Mid-Infrared Lifetime Imaging for Viability Evaluation of Lettuce Seeds Based on Time-Dependent Thermal Decay Characterization. Sensors 13, 2986-2996 (2013).
7. Navarro, P., Fernandez, C., Weiss, J. & Egea-Cortines, M. Development of a Configurable Growth Chamber with a Computer Vision System to Study Circadian Rhythm in Plants. Sensors 12, 15356-15375 (2012).
8. Kuriyama, S. & Rechnitz, G. A. Plant tissue-based bioselective membrane electrode for glutamate. Anal. Chim. Acta 131, 91-96 (1981).
9. Sidwell, J. S. & Rechnitz, G. A. “Bananatrode”: An electrochemical biosensor for dopamine. Biotechnol. Lett. 7, 419-422 (1985).
10. Kuriyama, S., Arnold, M. A. & Rechnitz, G. A. Improved membrane electrode using plant tissue as a biocatalyst. J. Memb. Sci. 12, 269-278 (1983).
11. Smit, N. & Rechnitz, G. A. Leaf based biocatalytic membrane electrodes. Biotechnol. Lett. 6, 209-214 (1984).
12. Manak, M. S., Paul, A.-L., Sehnke, P. C. & Ferl, R. J. REMOTE SENSING OF GENE EXPRESSION IN PLANTA: TRANSGENIC PLANTS AS MONITORS OF EXOGENOUS STRESS PERCEPTION IN EXTRATERRESTRIAL ENVIRONMENTS.
13. Oliphant, A. J. et al. Autumn olive (Elaeagnus umbellata) presence and proliferation on former surface coal mines in Eastern USA. Biol. Invasions 19, 179-195 (2017).
14. Jefferson, R. A., Burgess, S. M. & Hirsht, D. 18-Glucuronidase from Escherichia coli as a gene-fusion marker (DNA sequence/uidA gene/reporter gene/enzyme purification). Biochemistry 83, 8447-8451 (1986).
15. Jefferson, R. A., Kavanagh, T. A. & Bevan, M. W. GUS fusions: beta-glucuronidase as a sensitive and versatile gene fusion marker in higher plants. EMBO J. 6, 3901-7 (1987).
16. Michaelis, L. & Menton, M. I. Kinetics of invertase action. Biochem. Z. 49, 333-369 (1913).
17. Fishman, W. & Bernfeld, P. Glucuronidases. Methods Enzymol. (1955).
18. Kleber, H.-P. D. Schomburg and M. Salzmann (Editors), Enzyme Handbook Volume 1, Class 4: Lyases. 810 S., Loose-Leaf-Binder. Berlin-Heidelberg-New York-London-Paris-Tokyo-Hong Kong-Barcelona 1990. Springer-Verlag. DM 248,00. ISBN: 3-540-52579-3. J. Basic Microbiol. 31, 320-320 (1991).
19. Cottrell, F. Residual current in galvanic polarization regarded as a diffusion problem. Z. phys. Chem (1903).
20. Belkin, S. Microbial whole-cell sensing systems of environmental pollutants. Curr. Opin. Microbiol. 6, 206-212 (2003).
21. Felix, G. & Grosskopf, D. Rapid changes of protein phosphorylation are involved in transduction of the elicitor signal in plant cells. Proc. (1991).
22. Narasimhulu, S., Deng, X., Sarria, R. & Gelvin, S. Early transcription of Agrobacterium T-DNA genes in tobacco and maize. Plant Cell (1996).
23. Koncz, C., Olsson, O. & Langridge, W. Expression and assembly of functional bacterial luciferase in plants. Proc. (1987).
24. Horsch, R. & Rogers, S. Transgenic plants. Cold Spring Harb. (1985).
25. Vernick, S. et al. Electrochemical Biosensing for Direct Biopsy Slices Screening for Colorectal Cancer Detection. J. Electrochem. Soc. 158, P1 (2011).
26. Popovtzer, R., Neufeld, T., Ron, E. Z., Rishpon, J. & Shacham-Diamand, Y. Electrochemical detection of biological reactions using a novel nano-bio-chip array. Sensors Actuators B 119, 664-672 (2006).

1. A sensing system, comprising:
an electrochemical chip having an arrangement of electrodes configured for electrochemical sensing;
a microfluidic system having fluidic channels leading to ports on a surface of said sensing system, for delivering to a plant part a substrate for a reporter enzyme expressed by said plant; and
an attachment system for attaching said surface of the sensing system to a surface of said plant part in a manner that said fluidic ports contact said surface of said plant part.

2. A plant or part thereof comprising the sensing system of claim 1 attached thereto.

3. A method of sensing plant expression, comprising attaching the system of claim 1 to a plant part using said attachment system, and receiving a signal generated by said electrochemical chip in response to exposure to said substrate, thereby sensing said expression of said reporter enzyme by said plant.

4. The system, according to claim 1, wherein said surface of the sensing system is hydrophobic.

5. The system according to claim 1, wherein the sensing system comprises a micro-chamber for holding said substrate, and wherein said microfluidic system is constituted for delivering said substrate from said micro-chamber to said ports.

6. The system according to claim 5, wherein the sensing system comprises an inlet port for filling said micro-chamber with said substrate.

7. The system according to claim 1, wherein the sensing system comprises a controller for controlling dosage of said delivery of said substrate to said plant part.

8. The system according to claim 1, wherein the sensing system comprises a communication system for transmitting signals generated by said electrochemical chip over a communication network.

9. The system according to claim 8, wherein the sensing system comprises a controller for receiving control signals over said communication network via said communication system and controlling dosage of said delivery of said substrate to said plant part, based on said control signals.

10. The system according to claim 1, wherein said electrodes are deposited on said surface of the sensing system such that said when said surface of the sensing system is attached to said surface of said plant part, said electrodes contact said surface of said plant part.

11. The system according to claim 1, wherein said electrodes are beneath said surface of the sensing system, and wherein said microfluidic system is constituted to deliver said reporter enzyme from said ports to said electrochemical chip.

12. The system according to claim 1, wherein the reporter enzyme is under the transcriptional regulation of a regulatory element.

13. The system of claim 12, wherein said regulatory element is induced by abiotic or biotic stress.

14. The system of claim 1, wherein said reporter enzyme is heterologously expressed in said plant or part thereof.

15. A sensing system, comprising:
an electrochemical chip having an arrangement of electrodes configured for electrochemical sensing;
a microfluidic system having fluidic channels leading to ports on said surface, for receiving from said plant part a reporter enzyme and delivering said reporter enzyme to said electrochemical chip; and
an attachment system for attaching said surface of the sensing system to a surface of said plant part in a manner that said fluidic ports contact said surface of said plant part.

16. A plant or part thereof comprising the sensing system of claim 15 attached thereto.

17. A method of sensing plant expression, comprising attaching the system of claim 15 to a plant part using said attachment system, and receiving signals generated by said electrochemical chip in response to exposure of said electrodes to said reporter enzyme, thereby sensing said expression of said reporter enzyme by said plant.

18. The plant according to claim 16, wherein the reporter enzyme is under the transcriptional regulation of a regulatory element.

19. The plant of claim 18, wherein said regulatory element is induced by abiotic or biotic stress.

20. The plant of claim 16, wherein said reporter enzyme is heterologously expressed in said plant or part thereof.

21. A method of detecting a plant phenotype, the method comprising:
subjecting the plant of claim 16 to a stress condition of interest that is sensed by said regulatory element; and
sensing expression of said enzyme in response to said stress, said expression being indicative of the plant phenotype.

22. The system according to claim 15, wherein said surface of the sensing system is hydrophobic.

23. The system according to claim 15, wherein the sensing system comprises a communication system for transmitting signals generated by said electrochemical chip over a communication network.

24. The system according to claim 1, wherein said attachment system comprises at least one mechanical assembly selected from the group consisting of a clamp, a hook and loop and a snap.

25. The system according to claim 1, wherein said attachment system comprises an adhesive layer on said surface of the sensing system.

26. The system according to claim 1, wherein said electrochemical chip is flexible.

27. The system according to claim 1, wherein said electrochemical chip is attached to a surface said microfluidic system.

28. The system according to claim 1, wherein said electrodes are deposited on a surface said microfluidic system.

29. The system according to claim 1, wherein said electrochemical chip and said microfluidic system form a monolithic structure.

30. The system according to claim 1, wherein said plant part is not isolated plant cells.

31. The system according to claim 1, wherein said plant part is a leaf.

32. The system according to claim 1, wherein said plant part is a stem.

33. The system according to claim 1, wherein said plant part is a bud.

34. The system according to claim 1, wherein said plant part is a root.

35. The system according to claim 1, wherein said reporter enzyme can be any unique enzyme that is not naturally occurring in the plant.

36. The system according to claim 1, wherein said reporter enzyme can be naturally occurring in the plant in a lower concentration and overtly expressed by external stimulation.

37. The system according to claim 1, wherein said reporter enzyme is a beta glucoronidase.

38. The system according to claim 1, wherein said substrate is selected from the group consisting of a glucuronide, like Phenolphthalein-β-glucoronide and 4-aminophenyl-β-glucoronide.
